(19) United States
(12) Patent Application Publication
(10) Pub. No.: US 2009/0204148 A1
(43) Pub. Date: Aug. 13, 2009

(54) Adjustable Vertebral Body Elevator

(75) Inventors: Lawrence G. Lenke, St. Louis, MO (US); Eric Densford, West Memphis, AR (US); Rodney R. Ballard, Lakeland, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/027,545

(22) Filed: Feb. 7, 2008

(51) Int. Cl. A61B 17/58

(52) U.S. Cl. 606/246; 606/90

(57) ABSTRACT

An adjustable vertebral body elevator is disclosed and can include a handle and a spoon rotatably coupled to the handle by a locking assembly. The handle can rotate with respect to the spoon around an axis or rotation. Further, the locking assembly can be coaxial with the axis of rotation.

### FIELD OF THE DISCLOSURE

The present disclosure relates generally to surgical tools. More specifically, the present disclosure relates to vertebral body elevators.

### BACKGROUND

In human anatomy, the spine is a generally flexible column that can take tensile and compressive loads. The spine also allows bending motion and provides a place of attachment for keels, muscles and ligaments. Generally, the spine is divided into three sections: the cervical spine, the thoracic spine and the lumbar spine. The sections of the spine are made up of individual bones (vertebrae) that are separated from each other by intervertebral discs.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

During certain surgeries of the spine, e.g., a pedicle subtraction osteotomy, it may be necessary to protect the tissue in the area of the surgery to minimize the risk of injury, or further injury, to the patient. For example, it may be necessary to protect a patient's aorta and spinal cord during such a surgery.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan view of a vertebra;

FIG. 4 is a perspective view of an adjustable vertebral body elevator;

FIG. 5 is an exploded perspective view of an adjustable vertebral body elevator;

FIG. 6 is a front plan view of a handle associated with the adjustable vertebral body elevator;

FIG. 7 is a side plan view of the handle;

FIG. 8 is a front plan view of a spoon associated with the adjustable vertebral body elevator;

FIG. 9 is a side plan view of the spoon;

FIG. 10 is a side plan view of a push button associated with the adjustable vertebral body elevator;

FIG. 11 is a side plan view of a pin bushing associated with the adjustable vertebral body elevator;

FIG. 12 is a side plan view of a lock actuator pin associated with the adjustable vertebral body elevator;

FIG. 13 is a top plan view of a lock associated with the adjustable vertebral body elevator;

FIG. 14 is a detailed view of the adjustable vertebral body elevator taken at circle 14 in FIG. 4;

FIG. 15 is a detailed view of the adjustable vertebral body elevator taken at circle 15 in FIG. 5; and

FIG. 16 is a flow chart illustrating one method of using an adjustable vertebral body elevator.

### DETAILED DESCRIPTION OF THE DRAWINGS

An adjustable vertebral body elevator is disclosed and can include a handle and a spoon rotatably coupled to the handle by a locking assembly. The handle can rotate with respect to the spoon around an axis of rotation. Further, the locking assembly can be coaxial with the axis of rotation.

In another embodiment, an adjustable vertebral body elevator is disclosed and can include a handle that can have a proximal end and a distal end. The distal end of the handle can include a first collar. The adjustable vertebral body elevator can also include a spoon that can have a proximal end and a distal end. The proximal end of the spoon can include a second collar. Further, the second collar can abut the first collar. Also, the first collar and the second collar can be coaxial with each other and coaxial with an axis of rotation of the adjustable vertebral body elevator. The adjustable vertebral body elevator can also include a locking assembly at least partially installed within the first collar and the second collar. The locking assembly can be coaxial with the axis of rotation of the adjustable vertebral body elevator.

In yet another embodiment, a method of using an adjustable vertebral body elevator is disclosed and can include retrieving the adjustable vertebral body elevator. The adjustable vertebral body elevator can include a handle, a spoon, and a locking assembly connecting the handle and spoon. The handle can rotate with respect to the spoon along an axis of rotation and the locking assembly can be coaxial with the axis of rotation. The method can further include determining whether an angle between the handle and spoon is proper and moving the locking assembly to an unlocked position.

### Description of Relevant Anatomy

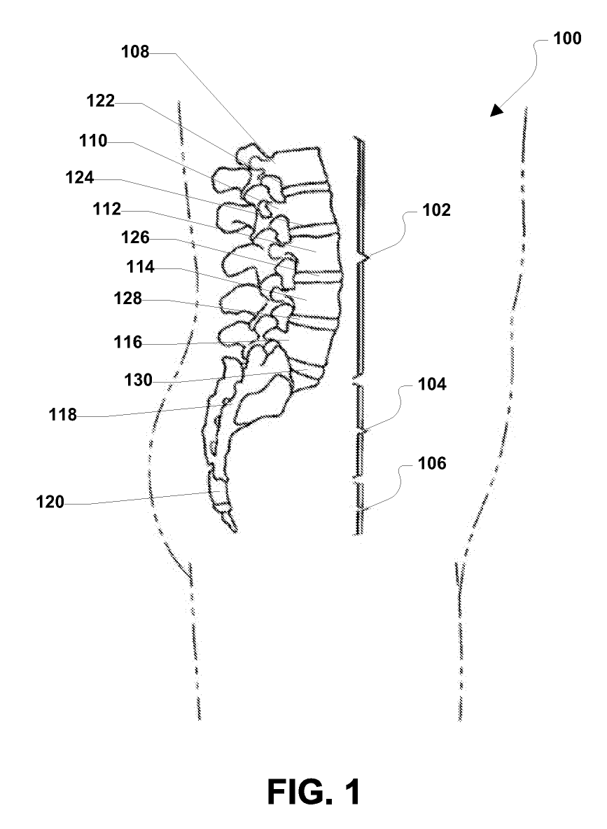

Referring initially to FIG. 1, a portion of a vertebral column, designated 100, is shown. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of repair, augmentation or treatment, that intervertebral lumbar disc 122, 124, 126, 128, 130 can be treated in accordance with one or more of the embodiments described herein.

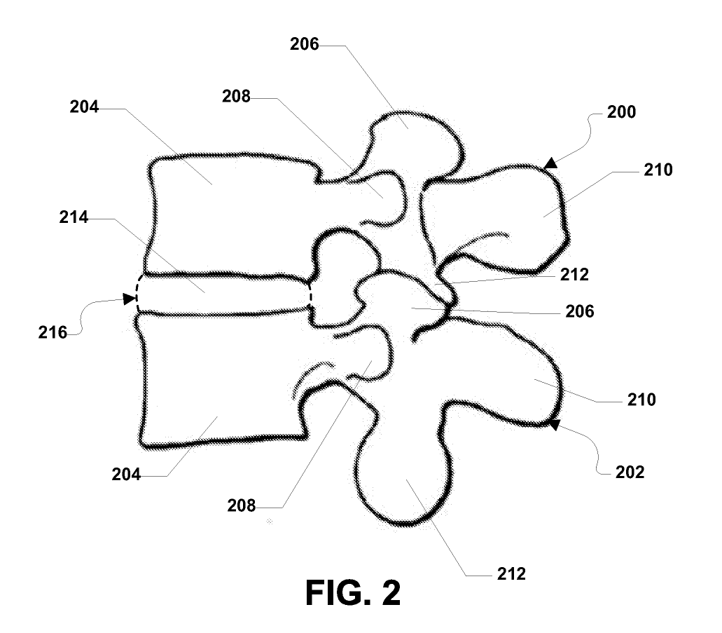

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral disc 216 between the superior vertebra 200 and the inferior vertebra 202.

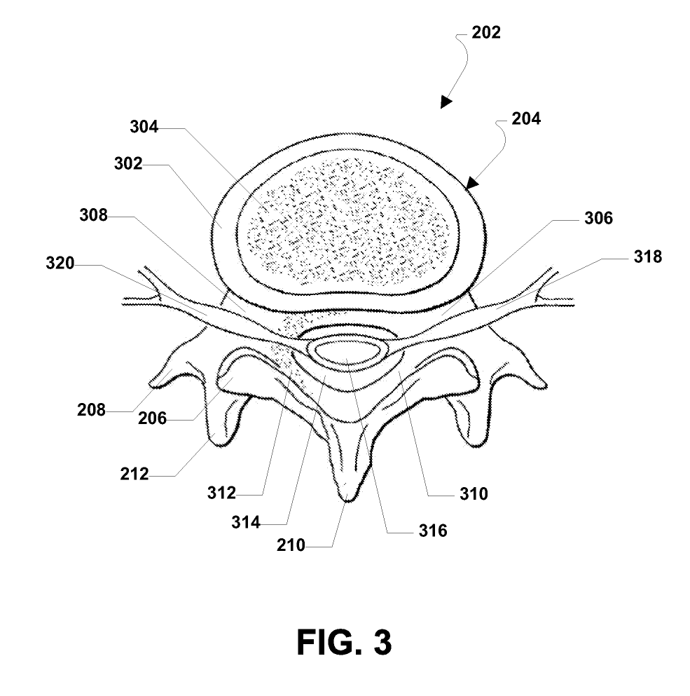

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

In order to correct certain spinal disorders, it may be necessary to install one or more implants along the spine. For example, scoliosis can be treated using a spinal fixation system. Further, a damaged disc can be replaced using a fusion device, a motion preserving implant, or a similar device. The installation of certain spinal devices may require the use of one or more bone screws to properly position the device and maintain the device in the proper position. The surgical screwdriver described herein may be used to install one or more surgical screws along the spinal column.

### Description of an Adjustable Vertebral Body Elevator

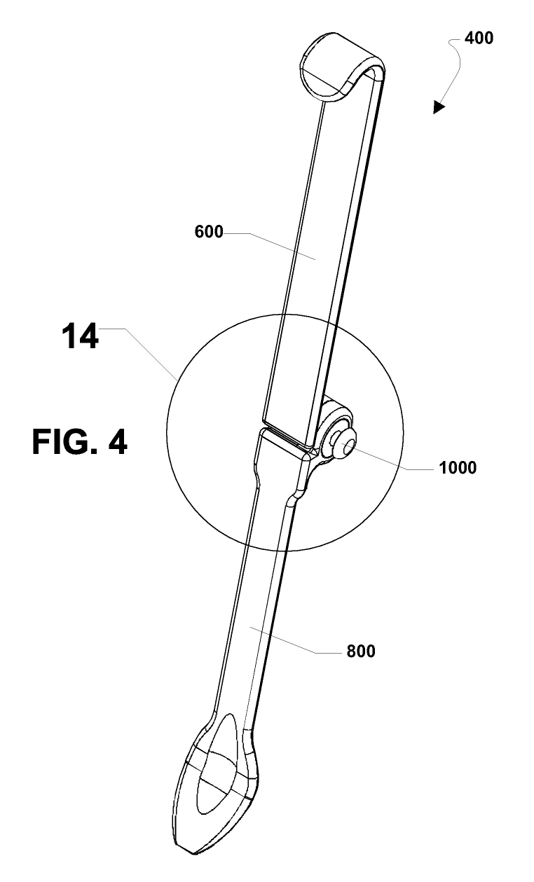

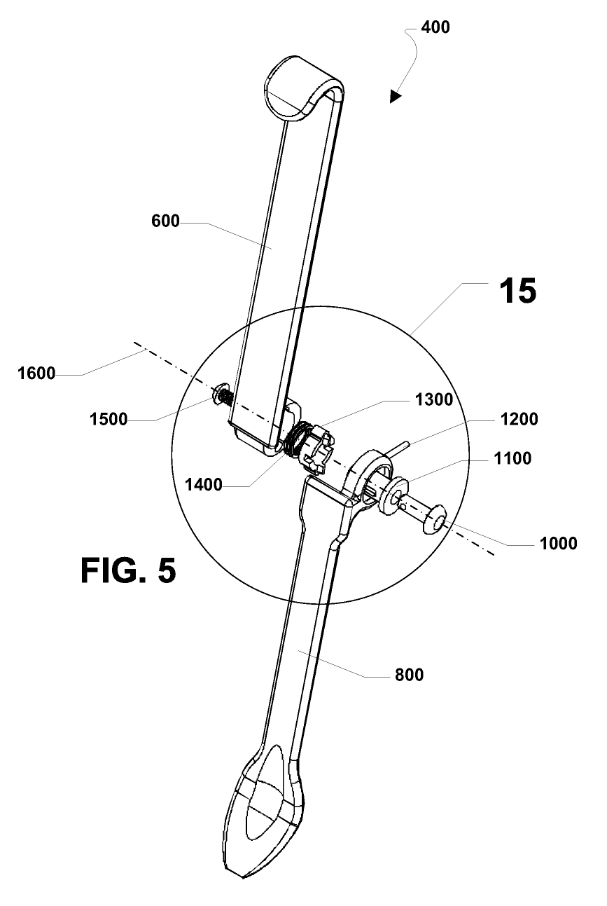

Referring to FIG. 4 and FIG. 5, an adjustable vertebral body elevator is shown and is generally designated 400. As shown, the adjustable vertebral body elevator 400 can include a handle 600 and a spoon 800. As described in further detail below, the handle 600 and the spoon 800 can be connected by a push button 1000 and a retractor pin bearing 1100. The push button 1000 and the retractor pin bearing 1100 can extend through the spoon 800 into the handle 600. A lock actuator pin 1200 can extend through the push button 1000. Further, a lock 1300 can be installed within the handle 600. The lock 1300 can extend partially into and engage the spoon 800. The adjustable vertebral body elevator 400 can also include a spring 1400 installed between the lock 1300 and the handle 600. The push button 1000, the retractor pin bearing 1100, the lock actuator pin 1200, the lock 1300, and the spring 1400 can be assembled as indicated in FIG. 5. Further, a bolt 1500 can extend through the handle 600 and threadably engage the push button 1000 in order to keep this assembly from coming apart.

As indicated in FIG. 5, the push button 1000, the pin bearing 1100, the lock 1300, the spring 1400, and the bolt 1500 can be substantially coaxial to each other along an axis of rotation 1600. Further, the handle 600 and the spoon 800 can rotate relative to each other around the axis of rotation 1600 that passes through the push button 1000, the pin bearing 1100, the lock 1300, the spring 1400, and the bolt 1500.

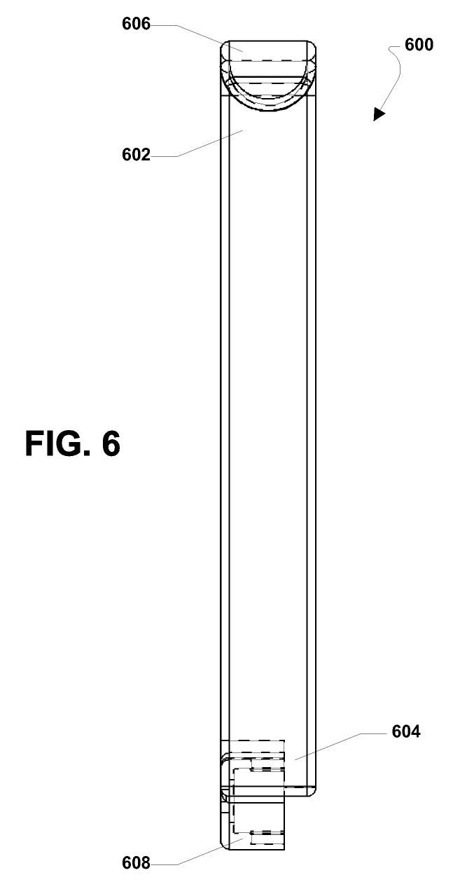

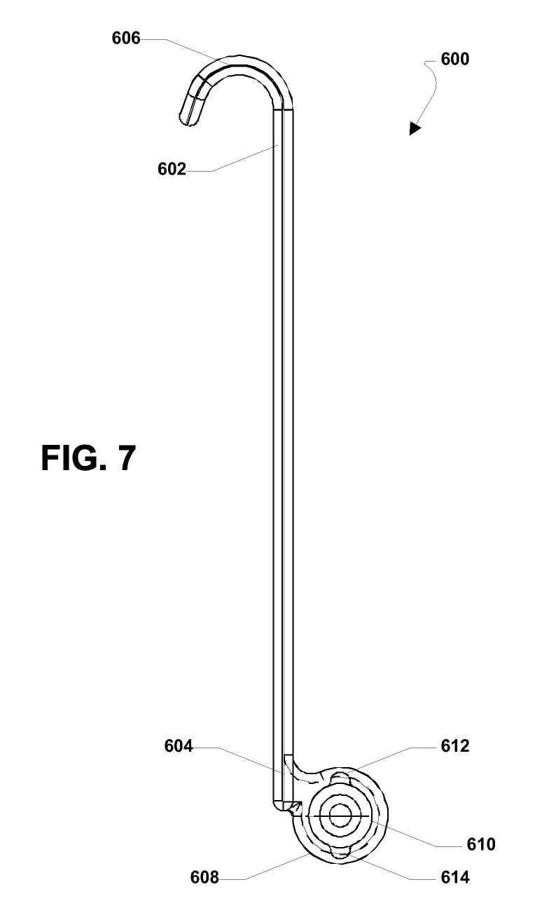

Referring to FIG. 6 through FIG. 13, details concerning the various parts of the adjustable vertebral body elevator 400 are illustrated. FIG. 6 and FIG. 7 illustrate the handle 600. As shown, the handle 600 can include a proximal end 602 and a distal end 604. The proximal end 602 of the handle 600 can include a curved portion 606 that can enhance a user's grip on the adjustable vertebral body elevator 400. Further, the distal end 604 of the handle 600 can include a first collar 608. The first collar 608 can be generally cylindrical and the first collar 608 can include a central bore 610. The first collar 608 can also include a first radial groove 612 and a second radial groove 614 that extend into the first collar 608 from the central bore 610. In a particular embodiment, the radial grooves 612, 614 are diametrically opposed to each other.

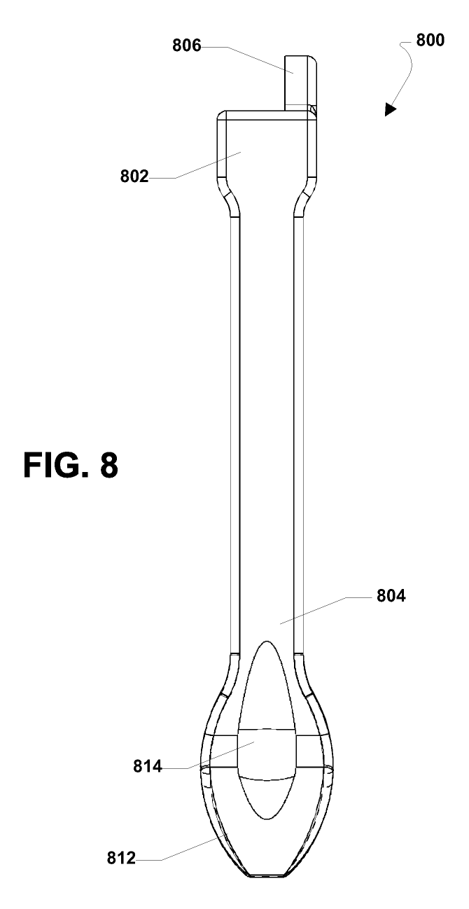

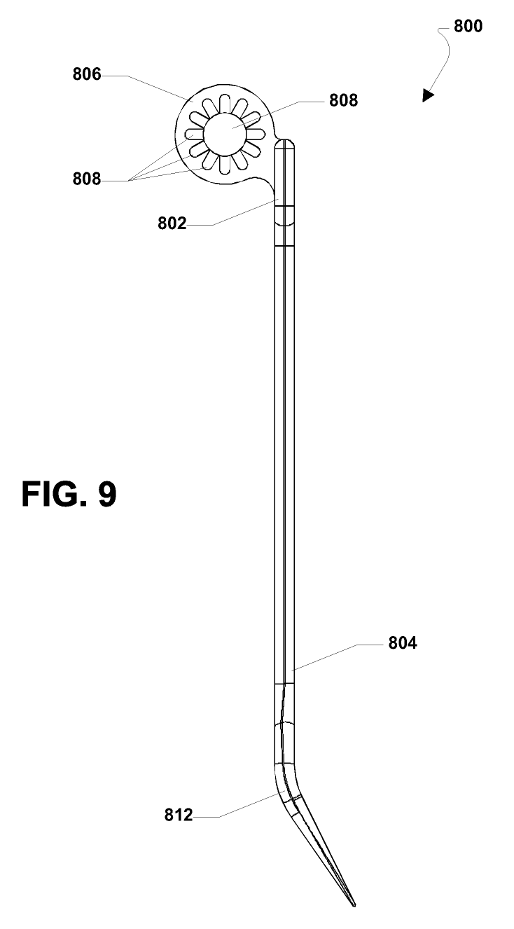

Referring to FIG. 8 and FIG. 9, the spoon 800 is depicted. The spoon 800 can include a proximal end 802 and a distal end 804. The proximal end 802 of the spoon 800 can include a second collar 608. The second collar 608 can be generally cylindrical and the second collar 608 can include a central bore 808. Moreover, the second collar 608 can include a plurality of radial grooves 810 that can extend into the second collar 608 from the central bore 808. In a particular embodiment, as shown, the second collar 608 can include twelve (12) radial grooves 810 with six (6) diametrically opposed pairs. However, the second collar 608 can include any other even number of grooves, e.g., four (4) grooves, six (6) grooves, eight (8) grooves, ten (10) grooves, fourteen (14) grooves, sixteen (16) grooves, eighteen (18) grooves, twenty (20) grooves, twenty-two (22) grooves, twenty-four (24) grooves, twenty-six (26) grooves, twenty-eight (28) grooves, thirty (30) grooves, thirty-two (32) grooves, thirty-four (34) grooves, thirty-six (36) grooves, thirty-eight (38) grooves, forty (40) grooves, forty-two (42) grooves, forty-four (44) grooves, forty-six (46) grooves, forty-eight (48) grooves, fifty grooves (50), fifty-two groves (52), fifty-four (54) grooves, fifty-six (56) grooves, fifty-eight (58) grooves, sixty (60) grooves, sixty-two (62) grooves, sixty-four (64) grooves, sixty-six (66) grooves, sixty-eight (68) grooves, seventy (70) grooves, seventy-two (72) grooves, etc.

FIG. 8 further shows that the distal end 804 of the spoon 800 can include an enlarged head 812. The enlarged head 812 can be generally elliptical. Further, the enlarged head 812 can be formed with a generally concave depression 814. During surgery of the spine, e.g., a pedicle subtraction osteotomy, the enlarged head 812 can help protect the anatomy in the surgical region. For example, the enlarged head 812 can help protect a patient's aorta and spinal cord during such a surgery.

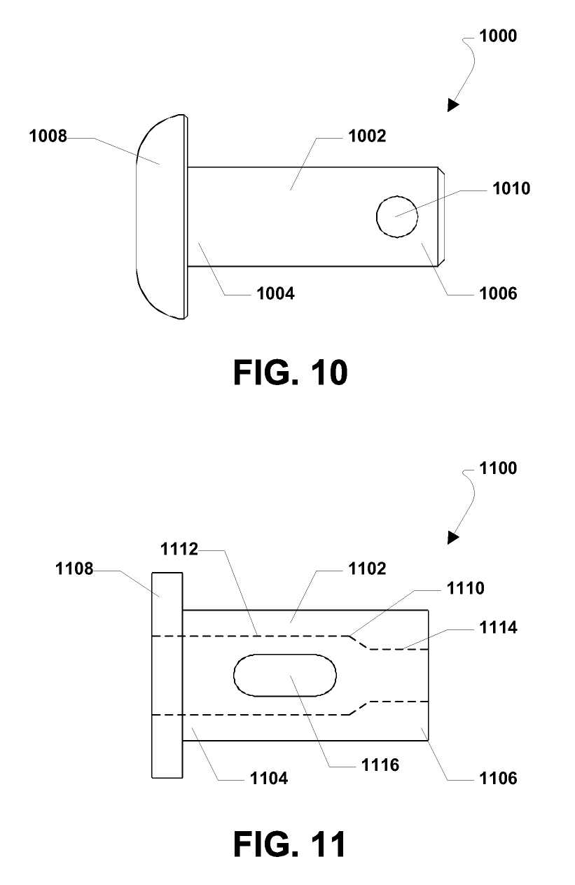

Referring now to FIG. 10, the push button 1000 is shown. As depicted, the push button 1000 can include a post 1002. The post 1002 can be solid and generally cylindrical. Further, the post 1002 can have a proximal end 1004 and a distal end 1006. FIG. 10 also shows that the push button 1000 can include a head 1008 attached to, or integrally formed with, the post 1002, e.g., the proximal end 1004 of the post 1002. Additionally, the push button 1000 can be formed with a lateral bore 1010 near the distal end 1010 of the post 1002.

FIG. 11 illustrates the details of the pin bearing 1100. As shown, the pin bearing 1100 can include a post 1102. The post 1102 can be hollow and generally cylindrical. Further, the post 1102 can have a proximal end 1104 and a distal end 1106. FIG. 11 also shows that the pin bearing 1100 can include a head 1108 attached to, or integrally formed with, the post 1102, e.g., the proximal end 1104 of the post 1102. The pin bearing 1100 can also include a longitudinal bore 1110 that can extend through the entire length of the pin bearing 1100—including the head 1108 of the pin bearing 1100. The longitudinal bore 1110 can include a smooth portion 1112 and a threaded portion 1114. As illustrated in FIG. 11, the pin bearing 1100 can include one or more slots 1116 that can extend through the post 1102 into the longitudinal bore 1100 formed there through.

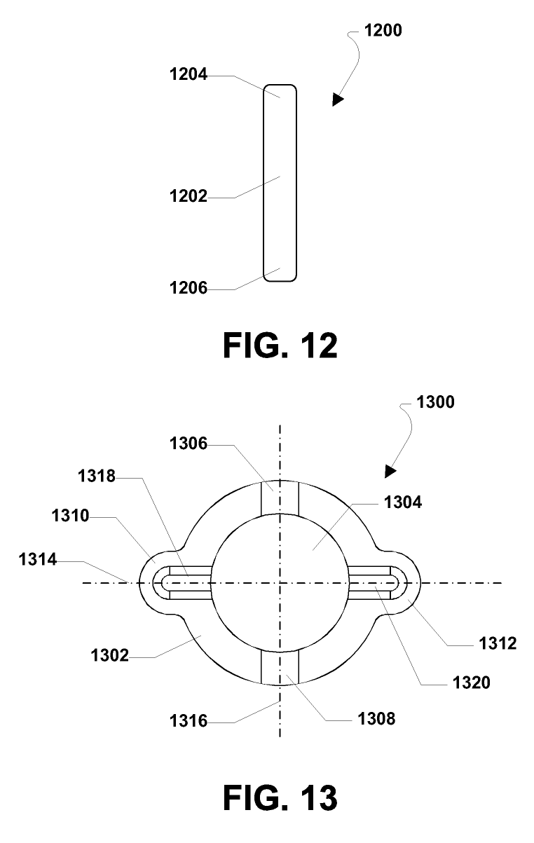

FIG. 12 shows the lock actuator pin 1200. As shown, the lock actuator pin 1200 can include a solid, generally cylindrical body 1202 having a proximal end 1204 and a distal end 1204.

Referring now to FIG. 13, details concerning the construction of the lock 1300 are depicted. FIG. 13 shows that the lock 1300 can include a generally cylindrical collar 1302 formed with a central bore 1304. A first radial slot 1306 and a second radial slot 1308 can extend through the wall of the collar 1302 into the central bore 1304. In a particular embodiment, the radial slots 1306, 1308 can be diametrically opposed to each other. As further shown in FIG. 13, the collar 1302 can include a first ear 1310 and a second ear 1312 that can extend from the periphery of the collar 1302. In a particular embodiment, the ears 1310, 1312 can be diametrically opposed to each other. Further, a first axis 1314 passing through the ears 1310, 1312 can be substantially perpendicular to second axis 1316 passing through the radial slots 1306, 1308.

FIG. 13 further illustrates a first locking tab 1318 that can extend from the first ear 1310. In a particular embodiment, the first locking tab 1318 can extend perpendicularly from the face of the collar 1302 in the area of the collar 1302 established by the first ear 1310. Also, a second locking tab 1320 can extend from the face of the collar 1302 in the area of the collar established by the second ear 1312. In a particular embodiment, the second locking tab 1320 can extend perpendicularly from the second ear 1312.

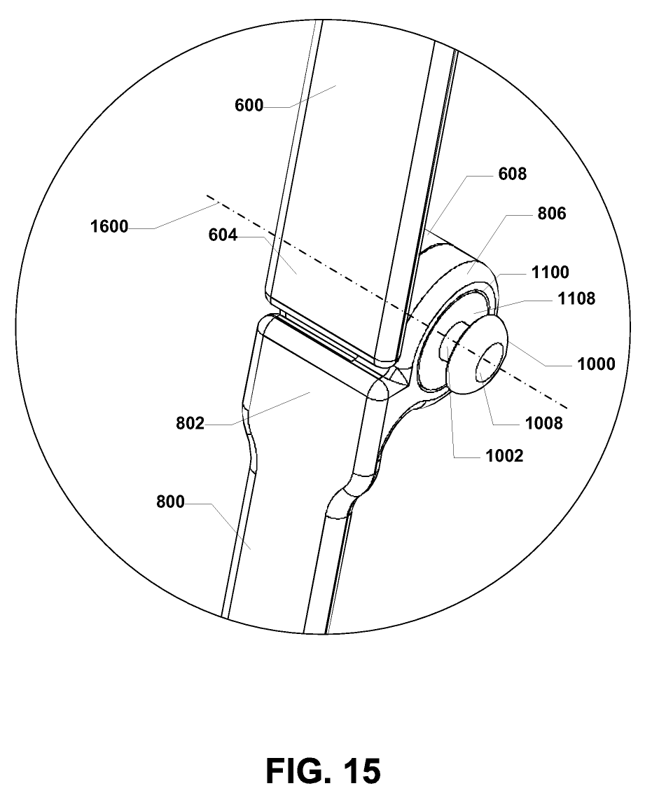

Referring to FIG. 15, the adjustable vertebral body elevator 400 can be assembled as described below. The spring 1400 can be placed within the bore 610 of the first collar 608 that extends from the distal end 604 of the handle 600. Thereafter, the lock 1300 can be placed into the bore 610 of the first collar 608. Specifically, the lock 1300 can be oriented within the bore 610 of the first collar 608 so that the first ear 1310 fits into and engages the first radial groove 612 within the first collar 608 and so that the second ear 1312 fits into and engages the second radial groove 614 within the first collar 608.

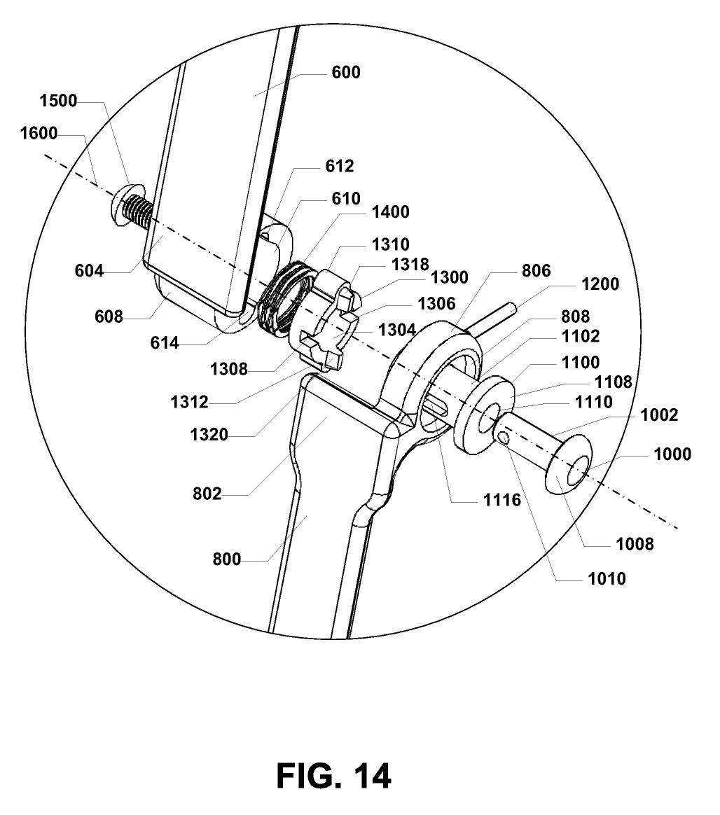

After the lock 1300 is installed within the first collar 608 of the handle 600, the spoon 800 can be engaged within the handle 600 so that the second collar 608 formed on the proximal end 802 of the spoon 806 abuts the first collar 608 of the handle 600. In a particular embodiment, the collars 608, 806 can be coaxial with each other. When the collars 608, 806 are abutted as described, the first locking tab 1318 and the second locking 1320 formed on the lock 1300 can engage an opposing pair of radial grooves (not shown in FIG. 14) formed in the second collar 608 on the spoon 800. As shown, the post 1102 of the pin bearing 1100 can be inserted through the central bore 808 formed in the second collar 608 of the spoon 800. The pin bearing 1100 can extend through the central bore 1304 formed in the lock 1300 and at least partially through the spring 1400 and at least partially into the central bore 610 formed in the first collar 608 of the handle 600. Further, the head 1108 of the pin bearing 1100 can fit into the central bore 808 formed in the second collar 608 of the spoon 800.

FIG. 14 indicates that the post 1002 of the push button 1000 can fit into the central bore 1110 formed in the pin bearing 1100. Once the post 1002 of the push button 1000 is installed within the pin bearing 1100, the lock actuator pin 1200 can be installed through the slots 1116 formed in the post 1102 of the pin bearing 1100 and through the lateral bore 1010 formed in the post 1002 of the push button 1000. The lock actuator pin 1200 can extend into and engage the radial slots 1306, 1308 formed in the lock 1300. In a particular embodiment, the components can be assembled, as described, to form a locking assembly. After, the locking assembly is assembled, the bolt 1500 can extend into the first collar 608 of the handle 600 and the bolt 1500 can be threadably engaged with the post 1102 of the pin bearing 1100.

In a particular embodiment, the push button 1000, the pin bearing 1100, the second collar 608 of the spoon 800, the lock 1300, the spring 1400, the first collar 608 of the handle 600, and the bolt 1500 are substantially coaxial with each other and the axis of rotation 1600 of the adjustable vertebral body elevator 400. Further, the push button 1000, the pin bearing 1100, the second collar 608 of the spoon 800, the lock 1300, the spring 1400, the first collar 608 of the handle 600, and the bolt 1500 can rotate about the axis of rotation 1600 of the adjustable vertebral body elevator 400. Moreover, the locking assembly is coaxial with the axis or rotation 1600 of the adjustable vertebral body elevator 400. The lock actuator pin 1200 can be perpendicular to the axis or rotation 1600 of the adjustable vertebral body elevator 400 and substantially centered around the axis or rotation 1600 of the adjustable vertebral body elevator 400. Accordingly, the lock actuator pin 1200 can also rotate about the axis of rotation 1600 of the adjustable vertebral body elevator 400.

FIG. 15 shows a close-up view of the adjustable vertebral body elevator 400 near the locking assembly. In a particular embodiment, the locking assembly can be moved between a locked configuration, shown in FIG. 15, and an unlocked configuration, not shown. In the locked configuration, the spring 1400 (not shown in FIG. 15) can bias the lock 1300 (not shown in FIG. 15) toward the second collar 608 on the proximal end 802 of the spoon 800. Further, in the locked configuration the locking tabs 1318, 1320 (not shown in FIG. 15) can engage an opposing pair of radial grooves 810 formed in the second collar 608 of the spoon 800 and the locking tabs 1318, 1320 can substantially prevent the spoon 800 from rotating with respect to the handle 600 around the locking assembly. Also, as shown in FIG. 15, in the locked configuration, the spring 1400 can bias the push button 1000 so that the push button 1000 extends out of the pin bearing 1100, along the axis of rotation 1600 of the adjustable vertebral body elevator 400, and so that the head 1008 head 1008 of the push button 1000 is slightly spaced from the head 1108 of the pin bearing 1100.

In a particular embodiment, to move the locking assembly to the unlocked configuration, the head 1008 of the push button 1000 can be pressed toward the head 1108 of the pin bearing 1100, along the axis of rotation 1600 of the adjustable vertebral body elevator 400, until the head 1008 of the push button 1000 contacts, or otherwise engages, the head 1108 of the pin bearing 1100. As the push button 1000 advances into the pin bearing 1100, the lock actuator pin 1200 can push the lock 1300 and bias the lock 1300 away from the second collar 608 on the spoon 800 until the locking tabs 1318, 1320 disengage the radial grooves 810 formed in the second collar 608 of the spoon 800. In the unlocked configuration, the spoon 800 can be rotated relative to the handle 600 until a desired angle between the spoon 800 and handle 600 is reached. Thereafter, the push button 1000 can be released and the locking assembly can return to the locked configuration in a new locked position.

In a particular embodiment, the adjustable vertebral body elevator 400 can be moved between a plurality of locked positions. For example, the vertebral body elevator 400 can be moved from a locked position in which the handle 600 is substantially co-linear with the spoon 800 and the angle between the handle 600 and the spoon is approximately equal to one-hundred and eighty degrees (180) to a locked position in which the handle 600 is angled with respect to the spoon 800.

In one embodiment, the adjustable vertebral body elevator 400 can be moved between two (2) positions that are spaced ninety degrees (90°) apart. In another embodiment, the adjustable vertebral body elevator 400 can be moved between three (3) positions that are spaced sixty degrees (60°) apart. In yet another embodiment, the adjustable vertebral body elevator 400 can be moved between four (4) positions that are spaced forty-five degrees (45°) apart. In still another embodiment, the adjustable vertebral body elevator 400 can be moved between six (6) positions that are spaced thirty degrees (30°) apart. In another embodiment, the adjustable vertebral body elevator 400 can be moved between twelve (12) positions that are spaced sixty degrees (15°) apart. In still yet another embodiment, the adjustable vertebral body elevator 400 can be moved between eighteen (18) positions that are spaced ten degrees (10°) apart. In yet still another embodiment, the adjustable vertebral body elevator 400 can be moved between thirty-six (36) positions that are spaced five degrees (5°) apart.

### Description of a Method of Using an Adjustable Vertebral Body Elevator

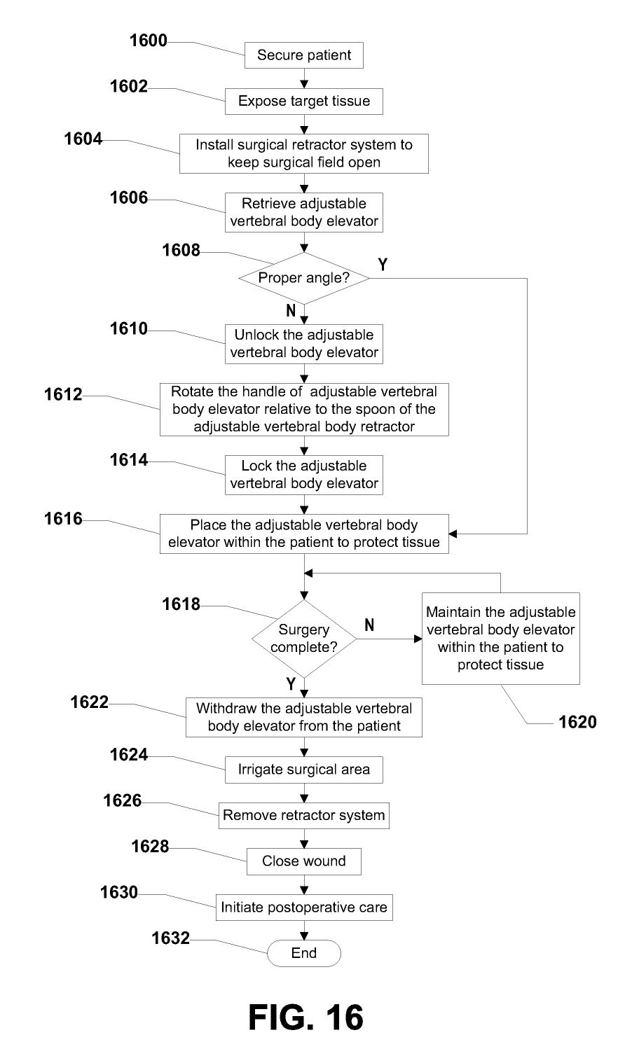

Referring to FIG. 16, a method of using an adjustable vertebral body elevator is shown and commences at block 1600. At block 1600, a patient can be secured on an operating table. For example, the patient can be secured in a prone position to allow a posterior approach to be used to access the patient's spinal column. Alternatively, the patient can be secured in a supine position to allow an anterior approach to be used to access the patient's spinal column. Further, the patient can be secured in a lateral decubitus position to allow a lateral approach to be used to access the patient's spinal column.

Moving to block 1602, the target tissue is exposed. Further, at block 1604, a surgical retractor system can be installed to keep the surgical field open. For example, the surgical retractor system can be a surgical retractor system configured for posterior access to a spinal column. Alternatively, the surgical retractor system can be a surgical retractor system configured for anterior access to a spinal column. Also, the surgical retractor system can be a surgical retractor system configured for lateral access to a spinal column.

Moving to block 1606, the adjustable vertebral body elevator can be retrieved. At decision step 1608, the user can determine whether the adjustable vertebral body elevator is configured with the proper angle for use with the patient. If the angle is improper, or incorrect, the method can proceed to block 1610 and the adjustable vertebral body elevator can be unlocked. The adjustable vertebral body elevator can be unlocked by pressing a push button on the adjustable vertebral body elevator, as described herein. Thereafter, at block 1612, the handle of the adjustable vertebral body elevator can be rotated relative to the spoon of the adjustable vertebral body elevator to establish a new angle. At block 1614, the adjustable vertebral body elevator can be locked, e.g., by releasing the push button.

Continuing to block 1616, the spoon of the adjustable vertebral body elevator can be placed within the patient to protect tissue within the patient. Returning to decision step 1608, if the adjustable vertebral body elevator is configured with the proper angle for use with the patient, the method can proceed directly to block 1616 and continue as described herein. From block 1616, the method can move to decision step 1618.

At decision step 1618, the user can determine whether the surgery is complete. If the surgery is not complete, the method can proceed to block 1620 and the spoon of the adjustable vertebral body elevator can be maintained within the patient to protect the tissue within the patient. Conversely, if the surgery is complete, the method can continue to block 1622 and the adjustable vertebral body elevator can be completely withdrawn from the patient. Moving to block 1624, the surgical space can be irrigated. Further, at block 1626, the retractor system can be removed. At block 1628, the surgical wound can be closed. The surgical wound can be closed using sutures, surgical staples, or any other surgical technique well known in the art. Moving to block 1630, postoperative care can be initiated. The method can end at state 1632.

### CONCLUSION

With the configuration of embodiments described above, an adjustable vertebral body elevator can be used to protect certain tissue within a patient during spinal surgeries. The adjustable vertebral body elevator can be adjusted to one of a plurality of positions to provide the best protection for different patients. For example, an adjustable vertebral body elevator can be rotated, or folded about the locking assembly and the adjustable vertebral body elevator can lie on top of the soft tissue that the adjustable vertebral body elevator is used to protect. Folding the adjustable vertebral body elevator can also improve visibility in the surgical field in which the adjustable vertebral body elevator is inserted and can allow more room for other tools used in the surgery. Further, the adjustable vertebral body elevator can be adjusted using one hand only, by pressing the push button, as described herein. Additionally, embodiments can be used for vertebral body exposure during vertebral column resections.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. An adjustable vertebral body elevator, comprising
a handle; and
a spoon rotatably coupled to the handle by a locking assembly, wherein the handle rotates with respect to the spoon around an axis of rotation and wherein the locking assembly is coaxial with the axis of rotation.

2. The adjustable vertebral body elevator of claim 1, wherein the locking assembly is movable between a locked configuration in which the locking assembly substantially prevents the spoon from rotating with respect to the handle and an unlocked configuration in which the spoon is rotatable relative to the handle.

3. The adjustable vertebral body elevator of claim 2, wherein the locking assembly comprises a push button lying along the axis of rotation of the adjustable vertebral body elevator.

4. The adjustable vertebral body elevator of claim 3, wherein the push button is movable along the axis of rotation in order to lock and unlock the locking assembly.

5. The adjustable vertebral body elevator of claim 1, wherein the adjustable vertebral body elevator is movable between a plurality of locked positions.

6. The adjustable vertebral body elevator of claim 5, wherein the adjustable vertebral body elevator is movable between a locked position in which the handle is substantially co-linear with the spoon and a locked position in which the handle is angled with respect to the spoon.

7. The adjustable vertebral body elevator of claim 6, wherein the adjustable vertebral body elevator is movable between two (2) positions that are spaced ninety degrees (90°) apart.

8. The adjustable vertebral body elevator of claim 6, wherein the adjustable vertebral body elevator is movable between three (3) positions that are spaced sixty degrees (60°) apart.

9. The adjustable vertebral body elevator of claim 6, wherein the adjustable vertebral body elevator is movable between four (4) positions that are spaced forty-five degrees (45°) apart.

10. The adjustable vertebral body elevator of claim 6, wherein the adjustable vertebral body elevator is movable between six (6) positions that are spaced thirty degrees (30°) apart.

11. The adjustable vertebral body elevator of claim 6, wherein the adjustable vertebral body elevator is movable between twelve (12) positions that are spaced sixty degrees (15°) apart.

12. The adjustable vertebral body elevator of claim 6, wherein the adjustable vertebral body elevator is movable between eighteen (18) positions that are spaced ten degrees (10°) apart.

13. The adjustable vertebral body elevator of claim 6, wherein the adjustable vertebral body elevator is movable between thirty-six (36) positions that are spaced five degrees (5°) apart.

14. The adjustable vertebral body elevator of claim 1, wherein the handle comprises a proximal end and a distal end, wherein the proximal end includes a hook and the distal end is coupled to the spoon.

15. The adjustable vertebral body elevator of claim 14, wherein the distal end of the handle includes a first collar coupled to the spoon.

16. The adjustable vertebral body elevator of claim 15, wherein the spoon comprises a proximal end and a distal end, wherein the proximal end of the spoon comprises a second collar that abuts the first collar.

17. The adjustable vertebral body elevator of claim 16, wherein the distal end of the spoon includes an enlarged head formed with a concave depression.

18. An adjustable vertebral body elevator, comprising:
a handle having a proximal end and a distal end, wherein the distal end comprises a first collar;
a spoon having a proximal end and a distal end, wherein the proximal end of the spoon comprises a second collar, wherein the second collar abuts the first collar and wherein the first collar and the second collar are coaxial with each other and coaxial with an axis of rotation of the adjustable vertebral body elevator; and
a locking assembly at least partially installed within the first collar and the second collar, wherein the locking assembly is coaxial with the axis of rotation of the adjustable vertebral body elevator.

19. The adjustable vertebral body elevator of claim 18, wherein the locking assembly is movable between a locked configuration in which the locking assembly substantially prevents the spoon from rotating with respect to the handle and an unlocked configuration in which the spoon is rotatable relative to the handle.

20. The adjustable vertebral body elevator of claim 19, wherein the locking assembly comprises:
a lock at least partially installed within the first collar; and
a spring installed between the lock and the first collar, wherein the spring biases the lock toward the second collar to engage the second collar and to substantially prevent the spoon from rotating with respect to the handle.

21. The adjustable vertebral body elevator of claim 20, wherein the locking assembly further comprises:
a pin bearing that extends through the second collar and the lock and at least partially into the first collar.

22. The adjustable vertebral body elevator of claim 21, wherein the locking assembly further comprises:
a push button that extends through the pin bearing, wherein the push button is movable along the axis of rotation in order to lock and unlock the locking assembly.

23. The adjustable vertebral body elevator of claim 22, wherein the locking assembly further comprises:
a lock actuator pin installed perpendicularly through a bore in a distal end of the push button, wherein the lock actuator pin engages the lock and as the push button is pushed into the pin bearing the lock actuator pin disengages the lock from the second collar so the handle is rotatable relative to the spoon.

24. A method of using an adjustable vertebral body elevator, the method comprising:
retrieving the adjustable vertebral body elevator having a handle, a spoon, and a locking assembly connecting the handle and spoon, wherein the handle rotates with respect to the spoon along an axis of rotation and wherein the locking assembly is coaxial with the axis of rotation;
determining whether an angle between the handle and spoon is proper; and
moving the locking assembly to an unlocked position.

25. The method of claim 24, further comprising:
rotating the handle with respect to the spoon about the locking assembly.

26. The method of claim 25, further comprising:
moving the locking assembly to a locked position in which the handle is locked with respect to the spoon.

27. The method of claim 24, wherein the locking assembly is moved to the unlocked position by sliding a push button of the locking assembly along the axis of rotation.
